(19) United States
(12) Patent Application Publication
(10) Pub. No.: US 2007/0088212 A1
(43) Pub. Date: Apr. 19, 2007

(54) MAGNETIC RESONANCE IMAGING APPARATUS

(76) Inventors: Naoyuki Takei, Tokyo (JP); Tetsuji Tsukamoto, Tokyo (JP)

Correspondence Address: Patrick W. Rasche;Armstrong Teasdale LLP, Suite 2600, One Metropolitan Square, St. Louis, MO, 63102, US

(21) Appl. No.: 11/550,071

(22) Filed: Oct. 17, 2006

(30) Foreign Application Priority Data
Oct. 19, 2005 (JP) 2005-304798

(51) Int. Cl. A61B 5/05; G01V 3/00

(52) U.S. Cl. 600/413; 324/318

(57) ABSTRACT

For the purpose of improving contrast of an image, and hence, image quality, a navigator sequence NS for acquiring navigator echo data is performed before performing an imaging sequence IS each time, and a displacement N of the diaphragm caused by respiratory motion is detected based on the navigator echo data. Then, an imaging sequence IS for acquiring imaging data in the imaged region is performed if the detected displacement N caused by respiratory motion N of the subject falls within an acceptance window AW. Thereafter, each of a plurality of sets of imaging data acquired in the imaging sequence IS performed a plurality of number of times is corrected using a correction factor corresponding to a time interval between a first imaging sequence IS1 in which each set of imaging data is acquired and a second imaging sequence IS2 performed before the first imaging sequence IS1, and a slice image is produced from the plurality of corrected sets of imaging data.

### CROSS REFERENCE TO RELATED APPLICATIONS

This application claims the benefit of Japanese Application No. 2005-304798 filed Oct. 19, 2005.

### BACKGROUND OF THE INVENTION

The present invention relates to a magnetic resonance imaging apparatus, and particularly to a magnetic resonance imaging apparatus for emitting an electromagnetic wave toward a subject to excite an imaged region in the subject in a static magnetic field space, conducting a scan to acquire magnetic resonance signals generated in the imaged region in the subject, and then producing an image of the subject based on the magnetic resonance signals acquired by conducting the scan.

Magnetic resonance imaging (MRI) apparatuses are widely used in various fields including a medical application and an industrial application.

A magnetic resonance imaging apparatus emits an electromagnetic wave toward a subject in a static magnetic field space to thereby excite spins of protons in the subject with a nuclear magnetic resonance (NMR) phenomenon, and conducts a scan to acquire magnetic resonance (MR) signals generated by the excited spins. Based on the magnetic resonance signals acquired in the scan, a slice image of a cross-sectional plane through the subject is produced.

In such imaging on a subject using the magnetic resonance imaging apparatus, if the subject moves during the scan, motion artifacts may appear in a produced slice image. For example, when the heart or abdomen of the subject is imaged, body motion such as respiratory or cardiac motion leads to development of motion artifacts and degenerates image quality.

To prevent such image quality degeneration due to motion artifacts, there is proposed methods of conducting imaging in synchronization with body motion such as respiratory or cardiac motion (see Patent Documents 1 and 2, for example).

[Patent Document 1] Japanese Patent Application Laid Open No. H10-277010

[Patent Document 2] Japanese Patent Application Laid Open No. 2002-102201

In such methods, a displacement caused by cyclic cardiac motion is detected as electrocardiographic signals, for example, and the magnetic resonance imaging apparatus repetitively scans the subject at a specific phase of cardiac motion of the subject based on the electrocardiographic signals. In the scan, first, a region containing the diaphragm, for example, is selectively excited to monitor respiratory motion of the subject, and a navigator sequence is performed to acquire magnetic resonance signals as navigator echo data. Subsequent to the navigator sequence, an imaging sequence is performed to acquire magnetic resonance signals as imaging data from a slice position at which a slice image is to be produced. At that time, if a displacement of the diaphragm obtained by the navigator sequence falls within a predefined acceptance window, the imaging data acquired by the subsequent imaging sequence is selected as raw data for the slice image to sequentially fill a k-space. In particular, since the heart rate of the subject is generally of the order of sixty beats per minute, the navigator sequence and imaging sequence are performed in a cycle of 1000 msec to acquire navigator echo data and imaging data, and imaging data that is acquired when a displacement of the diaphragm obtained by the navigator echo data falls within a predefined acceptance window is selected as raw data, which is for use as a material for a slice image. A slice image is then reconstructed based on the imaging data selected as raw data.

When imaging data is acquired while generating an RF pulse in a cycle of one second in synchronization with cardiac motion of the subject, however, longitudinal magnetization of protons in the imaged region is not fully recovered because the T1 value of the blood vessel is of the order of 1300 msec, for example, resulting in low signal intensity for imaging data acquired as described above. Thus, contrast of an image is sometimes lowered and it is difficult to improve image quality. Especially when the coronary artery is to be imaged, such an inconvenience is sometimes encountered.

### SUMMARY OF THE INVENTION

It is therefore an object of the present invention is to provide a magnetic resonance imaging apparatus capable of improving contrast of an image, and hence, image quality.

To attain the aforementioned object, the present invention provides a magnetic resonance imaging apparatus comprising: a scanning section performing a plurality of number of times an imaging sequence for emitting an electromagnetic wave toward a subject to excite an imaged region in said subject in a static magnetic field space, and acquiring magnetic resonance signals generated in said imaged region in said subject as a set of imaging data; and an image producing section for producing an image of said subject based on a plurality of said sets of imaging data acquired by said scanning section performing said imaging sequence, wherein: said magnetic resonance imaging apparatus further comprises a body motion detecting section for periodically detecting a displacement caused by body motion of said subject; said scanning section performs said imaging sequence if said displacement caused by body motion detected by said body motion detecting section falls within a specified range; and said image producing section corrects each of said plurality of sets of imaging data acquired in said imaging sequence performed a plurality of number of times by said scanning section using a correction factor corresponding to a time interval between a first imaging sequence in which each set of said imaging data is acquired and a second imaging sequence performed before said first imaging sequence, and then produces an image based on said plurality of corrected sets of imaging data.

According to the present invention, there is provided a magnetic resonance imaging apparatus capable of improving contrast of an image, and hence, image quality.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing the configuration of a magnetic resonance imaging apparatus 1 in an embodiment in accordance with the present invention.

FIG. 2 is a flow chart showing an operation in imaging the subject SU in the present embodiment.

FIG. 3 is a sequence chart depicting a sequence in scanning the subject SU in the present embodiment, wherein the horizontal axis represents a time axis t.

FIG. 4 is a pulse sequence chart depicting a navigator sequence NS in the present embodiment.

FIGS. 5a and 5b are diagrams showing the process of deciding whether a displacement N1 of the diaphragm falls within an acceptance window AW in the present embodiment.

FIG. 6 shows recovering longitudinal magnetization in the present embodiment.

### DETAILED DESCRIPTION OF THE INVENTION

An exemplary embodiment of the present invention will now be described with reference to the accompanying drawings.

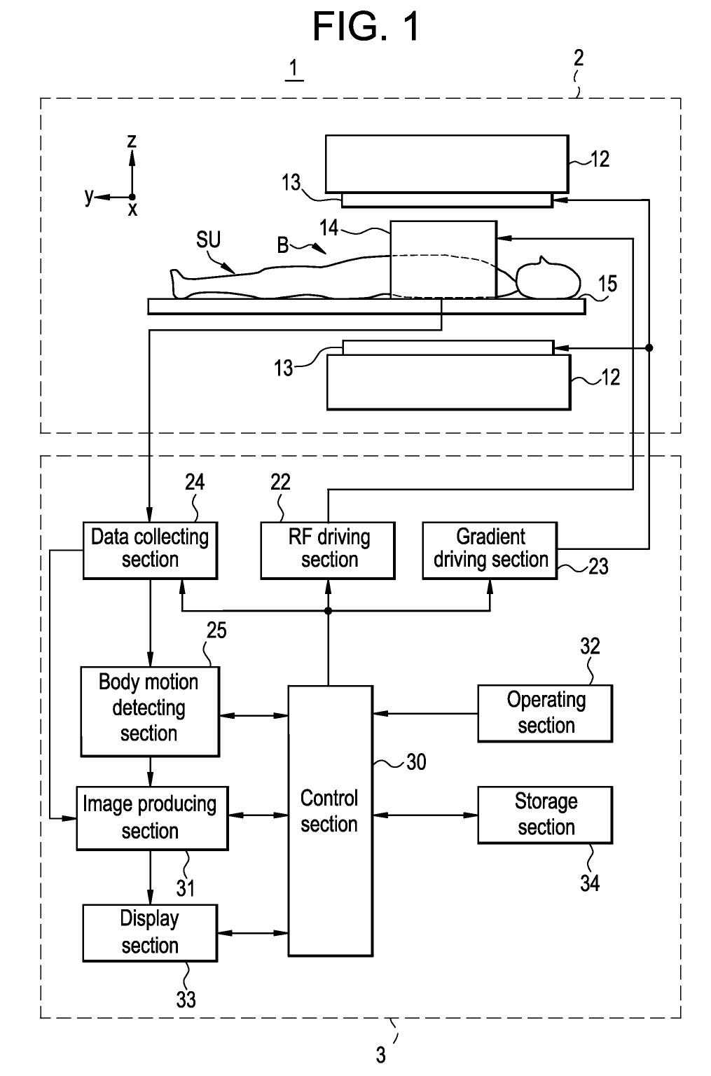

FIG. 1 is a block diagram showing the configuration of a magnetic resonance imaging apparatus 1 in an embodiment in accordance with the present invention.

As shown in FIG. 1, the magnetic resonance imaging apparatus 1 has a scanning section 2 and an operation console section 3.

Now the scanning section 2 will be described.

The scanning section 2 has a static magnetic field magnet section 12, a gradient coil section 13, an RF coil section 14, and a cradle 15, as shown in FIG. 1, for emitting an electromagnetic wave toward a subject SU to excite an imaged region in the subject SU in an imaging space B in which a static magnetic field is generated, and conducting a scan to acquire magnetic resonance signals generated in the imaged region in the subject SU.

In the present embodiment, the scanning section 2 repetitively scans the subject SU at a specific phase of cardiac motion of the subject SU based on electrocardiographic signals detected by a body motion detecting section 25, which will be described later, in the operation console section 3.

In the scan, first, a region containing the diaphragm in the subject SU is selectively excited to monitor respiratory motion of the subject SU, and a navigator sequence is performed to acquire magnetic resonance signals as navigator echo data. Subsequent to the navigator sequence, an imaging sequence is performed on a region containing the coronary artery in the subject SU as imaged region to acquire magnetic resonance signals as a set of imaging data for producing a slice image. Specifically, if a displacement of the diaphragm caused by respiratory motion detected by the body motion detecting section 25 based on the navigator echo data acquired by performing the navigator sequence falls within a prespecified range, the scanning section 2 performs the imaging sequence, detailed of which will be discussed later. In other words, the scanning section 2 repetitively performs an imaging sequence at the same phase of each cardiac cycle of the subject SU if a displacement of the diaphragm caused by respiratory motion of the subject SU falls within a prespecified range.

The components in the scanning section 2 will now be described one by one.

The static magnetic field magnet section 12 is comprised of, for example, a pair of permanent magnets to generate a static magnetic field in the imaging space B receiving the subject SU. The static magnetic field magnet section 12 here generates the static magnetic field such that the direction of the static magnetic field aligns with a direction Z that is perpendicular to the body axis direction of the subject SU. Alternatively, the static magnetic field magnet section 12 may be comprised of a superconductive magnet.

The gradient coil section 13 generates a gradient magnetic field in the imaging space B in which the static magnetic field is generated, to add spatial positional information to magnetic resonance signals received by the RF coil section 14. The gradient coil section 13 here is comprised of three coil systems of x-, y- and z-directions to generate gradient magnetic fields in a frequency encoding direction, a phase encoding direction, and a slice selective direction, depending upon imaging conditions. In particular, the gradient coil section 13 applies a gradient magnetic field in the slice selective direction of the subject SU to select a slice through the subject SU to be excited by the RF coil section 14 transmitting an RF pulse. The gradient coil 13 also applies a gradient magnetic field in the phase encoding direction of the subject SU to phase-encode magnetic resonance signals from the slice excited by the RF pulse. The gradient coil section 13 moreover applies a gradient magnetic field in the frequency encoding direction of the subject SU to frequency-encode magnetic resonance signals from the slice excited by the RF pulse.

The RF coil section 14 is disposed to surround the imaged region in the subject SU, as shown in FIG. 1. The RF coil section 14 transmits an RF pulse, which is an electromagnetic wave, to the subject SU in the imaging space B in which the static magnetic field is generated by the static magnetic field magnet section 12, to generate a high frequency magnetic field and excite spins of protons within the imaged region in the subject SU. The RF coil section 14 then receives an electromagnetic wave generated by the excited protons in the subject SU as magnetic resonance signals.

The cradle 15 has a table for laying thereon the subject SU. The cradle section 26 is moved between the inside and outside of the imaging space B based on a control signal from the control section 30.

Now the operation console section 3 will be described.

The operation console section 3 has an RF driving section 22, a gradient driving section 23, a data collecting section 24, the body motion detecting section 25, a control section 30, an image producing section 31, an operating section 32, a display section 33, and a storage section 34, as shown in FIG. 1.

The components in the operation console section 3 will now be described one by one.

The RF driving section 22 drives the RF coil section 14 to transmit an RF pulse to the imaging space B for generating a high frequency magnetic field. The RF driving section 22 modulates a signal from an RF oscillator into a signal of predetermined timing and envelope using a gate modulator based on a control signal from the control section 30, and then amplifies the signal modulated by the gate modulator at an RF power amplifier and outputs it to the RF coil section 14, thus transmitting the RF pulse.

The gradient driving section 23 applies a gradient pulse to the gradient coil section 13 and drives the section 13 based on a control signal from the control section 30, to generate a gradient magnetic field in the imaging space B in which the static magnetic field is generated. The gradient driving section 23 has three driving circuits (not shown) corresponding to the three systems of the gradient coil section 13.

The data collecting section 24 collects magnetic resonance signals received by the RF coil section 14 based on a control signal from the control section 30. The data collecting section 24 here has a phase detector that phase-detects magnetic resonance signals received by the RF coil section 14 with reference to the output from the RF oscillator in the RF driving section 22. Thereafter, an A/D converter is used to convert the magnetic resonance signals, which are analog signals, into digital signals and output them.

In the present embodiment, the data collecting section 24 outputs magnetic resonance signals acquired as a set of imaging data by an imaging sequence performed by the scanning section 2 to the image producing section 31 in the operation console 3. Moreover, the data collecting section 24 outputs magnetic resonance signals acquired as navigator echo data by a navigator sequence performed by the scanning section 2 to the body motion detecting section 25.

The body motion detecting section 25 has a computer and a program for causing the computer to execute predetermined data processing, and executes data processing for detecting a displacement caused by body motion of the subject SU each time the scanning section 2 performs an imaging sequence.

In the present embodiment, the body motion detecting section 25 detects a displacement caused by cardiac motion of the subject SU using an electrocardiograph.

Along with this operation, the body motion detecting section 25 periodically detects a displacement caused by body motion of the subject SU before the scanning section 2 performs an imaging sequence. The body motion detecting section 25 here repetitively detects a displacement of the diaphragm of the subject SU, which varies with respiratory motion before the imaging sequence, for each cardiac cycle of the subject SU at the same phase in that cardiac cycle. In particular, the body motion detecting section 25 detects a displacement of the diaphragm moved by respiratory motion before the scanning section 2 performs an imaging sequence based on navigator echo data acquired by the scanning section 2 performing a navigator sequence.

The control section 30 has a computer and a program for causing the relevant components to execute an operation corresponding to a predetermined scan using the computer, and controls the relevant components. The control section 30 here is supplied with operation data from the operating section 32, and based on the operation data supplied from the operating section 32, outputs for control a control signal to the RF driving section 22, gradient driving section 23, and data collecting section 24 to conduct a predetermined scan, and outputs for control a control signal to the body motion detecting section 25, image producing section 31, display section 33, and storage section 34.

The image producing section 31 has a computer and a program for causing the computer to execute predetermined data processing, and reconstructs a slice image for a slice through the subject SU based on a control signal from the control section 30. In the present embodiment, the image producing section 31 produces a slice image of the subject SU based on a plurality of sets of imaging data acquired by the scanning section 2 performing the imaging sequence. The image producing section 31 here corrects each of the plurality of sets of imaging data acquired in the imaging sequence performed a plurality of number of times by the scanning section 2 using a correction factor corresponding to a time interval between a first imaging sequence in which each set of imaging data is acquired and a second imaging sequence performed before the first imaging sequence. Thereafter, a slice image for the subject SU is reconstructed based on the plurality of corrected sets of imaging data. The image producing section 31 then outputs the reconstructed slice image to the display section 33.

The operating section 32 is comprised of operation devices such as a keyboard and a pointing device. The operating section 32 is supplied with operational data by the operator, and outputs the operational data to the control section 30.

The display section 33 is comprised of a display device such as a CRT, and displays an image on its display screen based on a control signal from the control section 30. For example, the display section 33 displays on its display screen a plurality of images of input fields for the operator to input operational data via the operating section 32. The display section 33 also receives from the image producing section 31 data for a slice image of the subject SU produced based on magnetic resonance signals from the subject SU, and displays the slice image on its display screen.

The storage section 34 is comprised of a memory, and stores several kinds of data. The storage device 33 has the stored data accessed by the control section 30 as needed.

Now an operation in imaging the subject SU using the magnetic resonance imaging apparatus 1 of the aforementioned embodiment in accordance with the present invention will be described hereinbelow.

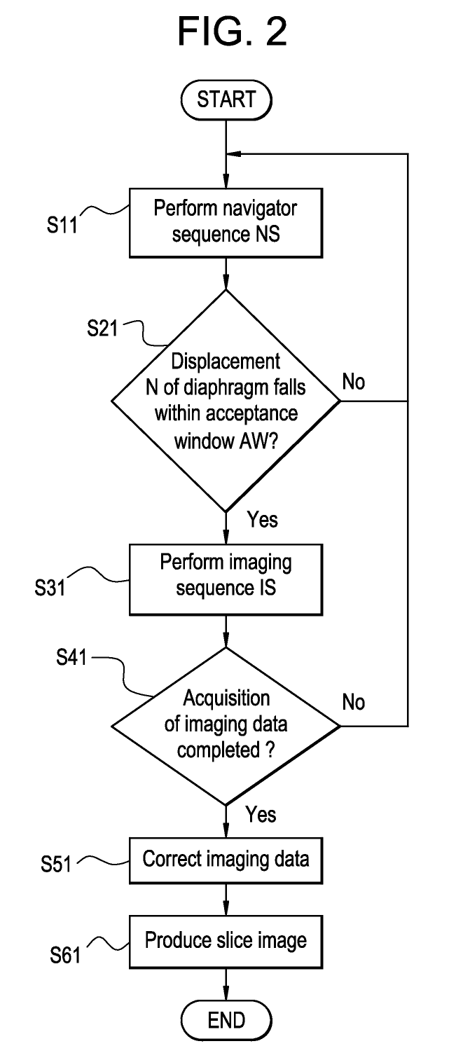

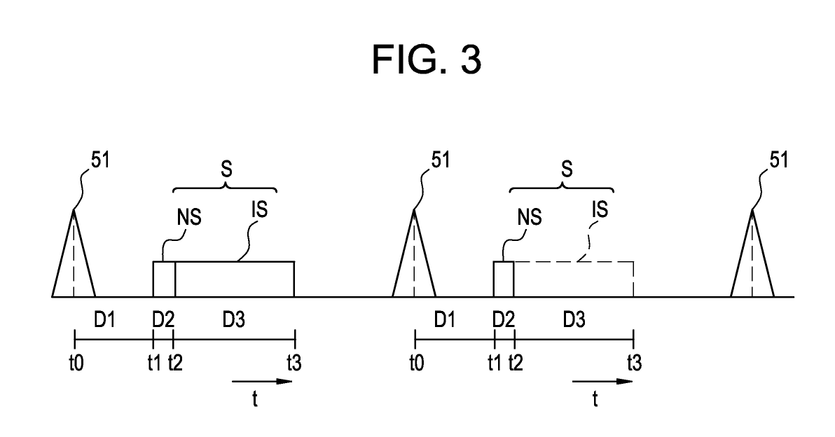

FIG. 2 is a flow chart showing an operation in imaging the subject SU in the present embodiment. FIG. 3 is a sequence chart depicting a sequence in scanning the subject SU in the present embodiment, wherein the horizontal axis represents a time axis t.

In the present embodiment, the scanning section 2 repetitively conducts a scan S on the subject SU at a specific phase of cardiac motion of the subject SU based on electrocardiographic signals detected by the body motion detecting section 25, to acquire magnetic resonance signals. In particular, as shown in FIG. 3, an R-wave 51 is detected in an electrocardiographic signal detected by the body motion detecting section 25, and the scanning section 2 periodically and repetitively starts the scan S on the thorax of the subject SU at a time point t1 corresponding to systole after a predetermined delay time D1 from a time point t0 at which the R-wave 51 is detected. For example, the scan S is repeated in a cycle of one second.

In conducting the scan S, a navigator sequence NS is initially performed (S11), as shown in FIGS. 2 and 3.

Specifically, to monitor respiratory motion of the subject SU, the scanning section 2 selectively excites spins in a region containing the diaphragm, and performs the navigator sequence NS to acquire magnetic resonance signals as navigator echo data according to a spin echo technique. For example, the navigator sequence NS is performed in a period from the time point t1 after the predetermined delay time D1 from the time point t0 at which the R-wave 51 is detected, to a time point t2 after a predetermined time D2 therefrom, as shown in FIG. 3.

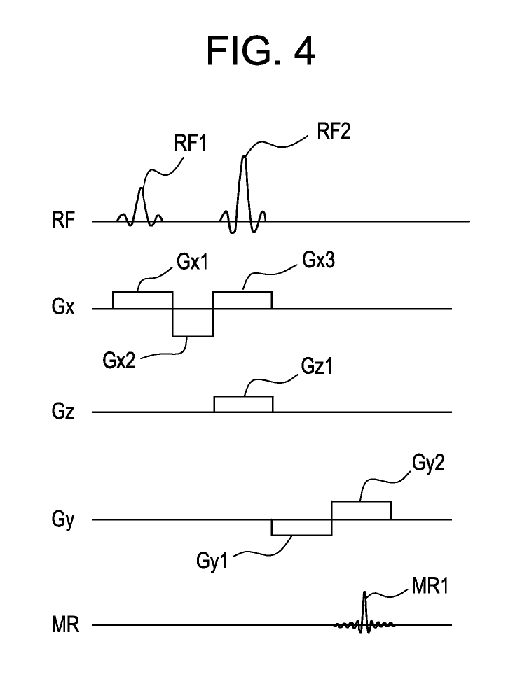

FIG. 4 is a pulse sequence chart depicting the navigator sequence NS. FIG. 4 shows an RF pulse RF, a gradient magnetic field Gx in an x direction, a gradient magnetic field Gz in a z direction, and a gradient magnetic field Gy in a y direction. In the drawing, the vertical axis represents intensity, and the horizontal axis represents a time axis.

In performing the navigator sequence NS, first, as shown in FIG. 4, a first x-gradient magnetic field Gx1 is applied along with a 90° pulse RF1 to thereby selectively 90°-excite a first slice plane containing the diaphragm of the subject SU. Thereafter, a second x-gradient magnetic field Gx2 is applied to the subject SU to rewind the phase, and a third x-gradient magnetic field Gx3 and a first z-gradient magnetic field Gz1 are applied along with a 180° pulse RF2 to thereby 180°-excite a second slice plane intersecting the first slice plane in the region containing the diaphragm. Then, first and second y-gradient magnetic fields Gy1 and Gy2 are applied for frequency encoding, and a magnetic resonance signal MR1 from a region at which the first slice plane intersects the second slice plane in the subject SU is acquired as navigator echo data.

The magnetic resonance signal MR1 acquired as navigator echo data by performing the navigator sequence NS is then collected by the data collecting section 24 and output to the body motion detecting section 25.

Next, a decision is made as to whether a displacement N of the diaphragm falls within an acceptance window AW (S21).

Specifically, the control section 30 decides whether the displacement N1 of the diaphragm of the subject SU detected by the body motion detecting section 25 falls within the acceptance window AW.

In particular, first, based on the navigator echo data acquired by the scanning section 2 performing the navigator sequence NS as described above, the body motion detecting section 25 determines a displacement N1 of the diaphragm moved by respiratory motion before the scanning section 2 performs the imaging sequence IS. The navigator echo data here is subjected to one-dimensional inverse Fourier transformation to generate a profile of the region containing the diaphragm, and a displacement N1 of the diaphragm is determined from the profile by the body motion detecting section 25. In the present embodiment, a portion in the generated profile that has high signal intensity corresponds to the abdomen, that having low signal intensity corresponds to the thorax, and a border portion between the portions representing the abdomen and thorax corresponds to the diaphragm; thus, a position at which the border portion corresponding to the diaphragm has moved in the body axis direction is determined as displacement N1 of the diaphragm by the body motion detecting section 25.

Thereafter, the control section 30 applies comparison processing on the displacement N1 of the diaphragm of the subject SU detected by the body motion detecting section 25 and upper and lower thresholds of the predefined acceptance window AW to decide whether the displacement N1 falls within the acceptance window AW.

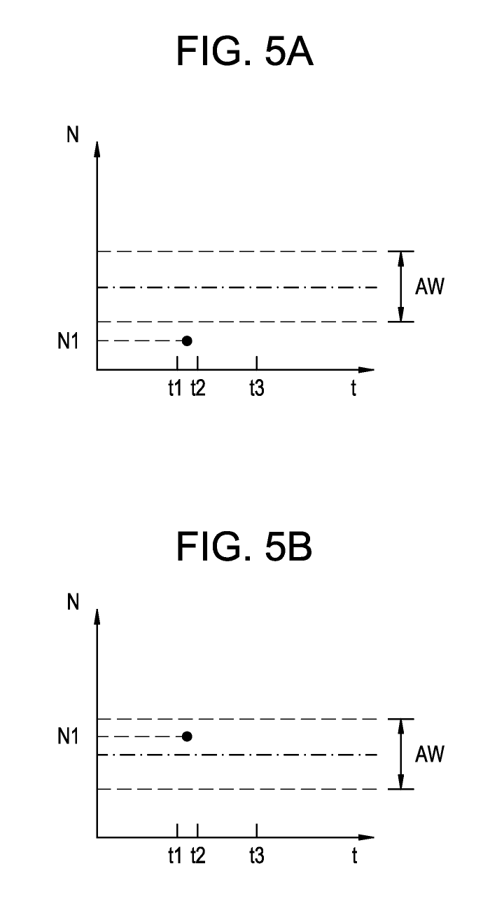

FIG. 5 is a diagram showing the process of deciding whether the displacement N1 falls within the acceptance window AW in the present embodiment, wherein the horizontal axis represents a time axis t, and the vertical axis represents a displacement N of the diaphragm. In the drawing, FIG. 5(a) shows the displacement N1 falling outside the acceptance window AW, and FIG. 5(b) shows the displacement N1 falling within the acceptance window AW.

When the displacement N1 of the diaphragm does not fall within the predefined acceptance window AW (No) as shown in FIG. 5(a), no imaging sequence IS is performed but a navigator sequence is performed in a next cardiac cycle (S11), as shown in FIG. 2.

On the other hand, if the displacement N1 of the diaphragm falls within the predefined acceptance window AW (Yes) as shown in FIG. 5(b), the imaging sequence IS is performed (S31), as shown in FIG. 2.

Specifically, subsequent to the navigator sequence NS, an imaging sequence is performed on the region containing the coronary artery in the subject SU as imaged region to acquire magnetic resonance signals as a set of imaging data for producing a slice image. For example, the scanning section 2 performs the imaging sequence IS according to a gradient echo technique. The imaging sequence IS is performed in a period from the time point t2 at which the navigator sequence NS is completed to a time point t3 after a predetermined time D3 therefrom, as shown in FIG. 3. The magnetic resonance signals acquired as a set of imaging data by performing the imaging sequence IS are then collected by the data collecting section 24.

Thus, in each scan S, if the displacement N1 of the diaphragm caused by respiratory motion detected by the body motion detecting section 25 based on the navigator echo data acquired by performing the navigator sequence NS falls within the predefined acceptance window AW, the scanning section 2 performs the imaging sequence IS, as indicated by a solid line on the left side of FIG. 3. On the other hand, in each scan S, if the displacement N1 of the diaphragm caused by respiratory motion detected by the body motion detecting section 25 based on the navigator echo data acquired by performing the navigator sequence NS does not fall within the predefined acceptance window, the scanning section 2 does not perform the imaging sequence IS, as indicated by a dashed line on the right side of FIG. 3. In other words, the scanning section 2 performs the imaging sequence IS at the same phase of each cardiac cycle of the subject SU if a displacement of the diaphragm caused by respiratory motion of the subject SU falls within a prespecified range.

Next, a decision is made as to whether acquisition of imaging data has been completed (S41), as shown in FIG. 2.

Specifically, the control section 30 decides whether imaging data corresponding to a matrix of a slice image to be produced have been collected by the data collecting section 24. For example, a decision is made as to whether imaging data corresponding to all phase encoding steps in the k-space have been acquired. If not all imaging data are collected by the data collecting section 24 (No), the control section 30 controls the relevant components to continue the scan S on the subject SU.

On the other hand, if all imaging data required have been collected by the data collecting section 25 and acquisition is completed (Yes), the acquired imaging data is subjected to correction (S51), as shown in FIG. 2.

Specifically, each of the plurality of sets of imaging data I acquired by the scanning section 2 performing the imaging sequence IS a plurality of number of times is corrected by the image producing section 31 using a correction factor Rpq corresponding to a time interval q between a first imaging sequence IS1 in which each set of imaging data I is acquired and a second imaging sequence IS2 performed before the first imaging sequence IS1. In other words, the image producing section 31 corrects variation in signal intensity among the plurality of sets of imaging data I caused by different recovery times for longitudinal magnetization Mz in the imaging sequence IS performed a plurality of number of times by the scanning section 2.

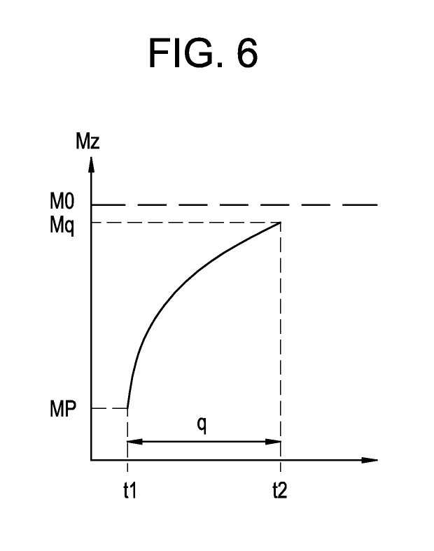

FIG. 6 shows recovering longitudinal magnetization in the present embodiment, wherein the vertical axis represents strength of longitudinal magnetization Mz, and the horizontal axis represents a time t.

As shown in FIG. 6, since longitudinal magnetization of protons is recovered according to EQ. (1) given below after signal acquisition, a correction factor Rpq is defined as EQ. (2) given below in the present embodiment, and a set of imaging data I acquired in a first imaging sequence IS1 is multiplied by the correction factor Rpq by the image producing section 31 according to EQ. (3) to obtain a corrected set of imaging data H. In the equations given below, Mp represents strength of longitudinal magnetization excited in a second imaging sequence IS2 before performing the first imaging sequence IS1, indicating strength of longitudinal magnetization at a first time point t1 after a recovery time of p seconds, as shown in FIG. 6. Mq represents strength of longitudinal magnetization at the start of excitation in the first imaging sequence IS1, indicating strength of longitudinal magnetization at a second time point t2 after a recovery time of q seconds from the first time point t1. Mo represents initial magnetization, and T1 represents the longitudinal relaxation time of an artery contained in the imaged region.

$$\begin{matrix}Mq = Mp \cdot exp ( - q / T 1 ) + Mo \cdot ( 1 - exp ( - q / T 1 ) ) & ( 1 ) \\ Rpq = Mq / Mo = [ Mp \cdot exp ( - q / T 1 ) + Mo \cdot ( 1 - exp ( - q / T 1 ) ) ] / Mo & ( 2 ) \\ H = Rpq \cdot I & ( 3 )\end{matrix}$$

Next, a slice image is produced (S61), as shown in FIG. 2.

Specifically, the image producing section 31 reconstructs a slice image of the subject SU using the plurality of sets of imaging data corrected as described above. The image producing section 31 then outputs the reconstructed slice image to the display section 33.

As described above, according to the present embodiment, the scanning section 2 performs a plurality of number of times an imaging sequence IS for emitting an electromagnetic wave toward a subject SU to excite an imaged region containing the coronary artery in the subject SU in the static magnetic field space, and acquiring magnetic resonance signals generated in the imaged region in the subject SU as a set of imaging data. At that time, before performing the imaging sequence IS each time, the scanning section 2 performs a navigator sequence NS for acquiring magnetic resonance signals from a region containing the diaphragm of the subject SU as navigator echo data, and the body motion detecting section 25 detects a displacement N of the diaphragm caused by respiratory motion based on the navigator echo data acquired by the scanning section 2 performing the navigator sequence NS. If the displacement N caused by respiratory motion of the subject SU detected by the body motion detecting section 25 falls within an acceptance window AW, the scanning section 2 performs the imaging sequence IS on a region containing the coronary artery in the subject SU as imaged region.

Thereafter, the image producing section 31 produces a slice image of the subject SU based on a plurality of sets of imaging data acquired by the scanning section 2 performing the imaging sequence IS. At that time, the image producing section 31 corrects each of the plurality of sets of imaging data acquired in the imaging sequence IS performed a plurality of number of times by the scanning section 2 using a correction factor corresponding to a time interval between a first imaging sequence IS1 in which each set of imaging data is acquired and a second imaging sequence IS2 performed before the first imaging sequence IS1. Then, the image producing section 31 produces a slice image of the subject SU based on the plurality of corrected sets of imaging data.

Thus, according to the present embodiment, since longitudinal magnetization of protons in an imaged region can be fully recovered by performing an imaging sequence in response to respiratory motion of the subject SU to acquire imaging data, and the imaging data is corrected to come close to signal intensity that would be obtained by fully recovered longitudinal magnetization according to a time interval between times at which an imaging sequence is performed, imaging data can be acquired as raw data having high signal intensity. Therefore, the present embodiment is capable of improving contrast of an image, and hence, image quality.

It should be noted that the magnetic resonance imaging apparatus 1 in the embodiment above corresponds to the magnetic resonance imaging apparatus of the present invention. The scanning section 2 in the embodiment above corresponds to the scanning section of the present invention. The body motion detecting section 25 in the embodiment above corresponds to the body motion detecting section of the present invention. The image producing section 31 in the embodiment above corresponds to the image producing section of the present invention. Finally, the display section 33 in the embodiment above corresponds to the display section of the present invention.

The present invention is not limited to being practiced in the aforementioned embodiment, and several variations may be employed.

For example, the navigator sequence may be performed according to any one of various imaging techniques, besides the spin echo technique.

Moreover, for example, body motion of the subject is not limited to being detected by performing a navigator sequence. For example, respiratory motion may be detected by fitting a belt around the thorax of the subject and detecting extension/contraction of the belt.

Many widely different embodiments of the invention may be configured without departing from the spirit and the scope of the present invention. It should be understood that the present invention is not limited to the specific embodiments described in the specification, except as defined in the appended claims.

1. A magnetic resonance imaging apparatus comprising: a scanning device performing a plurality of number of times an imaging sequence for emitting an electromagnetic wave toward a subject to excite an imaged region in said subject in a static magnetic field space, and acquiring magnetic resonance signals generated in said imaged region in said subject as a set of imaging data; and an image producing device for producing an image of said subject based on a plurality of said sets of imaging data acquired by said scanning device performing said imaging sequence, wherein:
said magnetic resonance imaging apparatus further comprises a body motion detecting device for periodically detecting a displacement caused by body motion of said subject;
said scanning device performs said imaging sequence if said displacement caused by body motion detected by said body motion detecting device falls within a specified range; and
said image producing device corrects each of said plurality of sets of imaging data acquired in said imaging sequence performed a plurality of number of times by said scanning device using a correction factor corresponding to a time interval between a first imaging sequence in which each set of said imaging data is acquired and a second imaging sequence performed before said first imaging sequence, and then produces an image of said subject based on said plurality of corrected sets of imaging data.

2. The magnetic resonance imaging apparatus of claim 1, wherein:
said body motion detecting device detects a displacement caused by respiratory motion of said subject.

3. The magnetic resonance imaging apparatus of claim 2, wherein:
said body motion detecting device detects said displacement caused by respiratory motion for each cardiac cycle of said subject.

4. The magnetic resonance imaging apparatus of claim 3, wherein:
said body motion detecting device repetitively detects said displacement caused by respiratory motion at the same phase over cardiac cycles of said subject; and
said scanning device performs said imaging sequence at the same phase over cardiac cycles of said subject.

5. The magnetic resonance imaging apparatus of claim 1, wherein:
said scanning device performs said imaging sequence on a region containing the coronary artery of said subject as said imaging region.

6. The magnetic resonance imaging apparatus of claim 1, wherein:
said scanning device performs a navigator sequence for acquiring said magnetic resonance signal as navigator echo data, before performing said imaging sequence; and
said body motion detecting device detects said displacement caused by body motion based on said navigator echo data acquired by said scanning device performing said navigator sequence.

7. The magnetic resonance imaging apparatus of claim 6, wherein:
said scanning device performs said navigator sequence to acquire said navigator echo data on a region containing the diaphragm in said subject.

8. The magnetic resonance imaging apparatus of claim 1, further comprising:
a display device for displaying on its display screen an image of said subject produced by said image producing device.
